(19) United States
(12) Patent Application Publication
(10) Pub. No.: US 2026/0060839 A1
(43) Pub. Date: Mar. 5, 2026

(54) Nosebleed Mitigating Hypothermic Bag

(71) Applicants: Akaki Gurjishvili, Brooklyn, NY (US); Nikoloz Chanturia, Tbilisi (GE)

(72) Inventors: Akaki Gurjishvili, Brooklyn, NY (US); Nikoloz Chanturia, Tbilisi (GE)

(21) Appl. No.: 19/242,628

(22) Filed: Jun. 18, 2025

Related U.S. Application Data
(60) Provisional application No. 63/689,530, filed on Aug. 30, 2024.

(51) Int. Cl. A61F 7/10; A61F 7/00; A61F 13/20; A61F 13/551

(52) U.S. Cl. CPC A61F 7/106; A61F 13/2005; A61F 2007/0006; A61F 2013/55195

(57) ABSTRACT

A system and method for stopping nosebleeds quickly and in a sanitary manner. The system comprises a layered bag, a cotton swab, and a disposable container. Within the layered bag is a cushion submerged in water. A water-reactive substance, namely urea, is stored within the cushion, designed to rapidly cool the layered bag after the cushion is ruptured. A method for using the device comprises the steps of attaching the layered bag to the nose bridge of the user and inserting the cotton swab into the nostril. Next, the user activates the urea by squeezing the cushion until the cushion ruptures, thereby dispersing the urea into the water. After the nosebleed stops, the user detaches the layered bag from the face and removes the contaminated cotton swab from the nostril. Next, the user inserts the contaminated cotton swab into the disposable container and then discards the disposable container.

### FIELD OF THE INVENTION

The present invention relates generally to a hypothermic bag for stopping nosebleeds. More specifically, the present invention is a layered bag with an integrated cushion that rapidly cools when activated.

### BACKGROUND OF THE INVENTION

In medical practice, first aid for nosebleeds follows standard approaches, with the primary method being the application of a cold object to the nasal bone for 10-15 minutes. This often involves placing an ice cube wrapped in cloth or using frozen liquids contained in plastic containers. However, these methods can result in freezing of the contact area, causing damage to the skin tissue, specifically the epidermis, at varying depths. An instant cold bag to be placed on the nasal bone is not currently in practice due to the potential damage. To clean the discharge of drained blood, dry and wet wipes made from various fabrics are commonly used. Fixation of these means to stop the bleeding is typically performed by the victim themselves, which can be distressing and lead to psycho-emotional destabilization. Easily accessible in any environment.

The use of ice often leads to hypothermic damage to the skin (epidermis). Moreover, during the cleaning of blood drainage, maintaining sanitary and hygienic conditions is crucial. There is a risk of the rescuer and bystanders coming into contact with the drained blood, which increases the potential spread of blood-borne diseases. During the care phase, it is essential to dispose of used cleaning agents in a designated bio-safe bag or container to ensure proper sanitation and safety.

An objective of the present invention is to provide users with a device that stops nosebleeds and provides a safe disposal system. The present invention intends to provide users with a device that attaches to the nasal bone and induces vasoconstriction within the nasal cavity. The present invention is activated by pressure, which can be performed by a young child, wherein the device then takes the shape and conforms to the nasal bone, increasing the effectiveness. In order to accomplish this, a preferred embodiment of the present invention comprises a layered bag and a cushion. Thus, the present invention is a system and method for stopping nosebleeds and facilitating proper disposal of contaminated cleaning agents without damaging the epidermis.

### SUMMARY OF THE INVENTION

The present invention is an instant cold pack that conforms to the nasal bone to stop nosebleeds. The present invention seeks to achieve vasoconstriction within the nasal cavity which stops a nosebleed quickly, absorbs blood drained from the nasal cavity using cotton balls, works without refrigeration, reduces skin tissue injuries, and provides psycho-emotional stabilization for the victim. To accomplish these objectives, the present invention comprises a layered bag that holds water and a cushion. When squeezed and punctured, the cushion releases a water-reactive substance (urea) that initiates an endothermic reaction, thereby cooling the water within the layered bag. After use, the cotton balls can be discarded in a two-part disposable container designed for safely discarding biohazardous material. Thus, the present invention is a system and method for stopping nosebleeds and facilitating proper disposal of contaminated cleaning agents without damaging the epidermis.

### BRIEF DESCRIPTION OF THE DRAWINGS

FIG. 1 is a top front perspective view of the present invention.

FIG. 2 is a bottom rear perspective view of the present invention.

FIG. 3 is a front elevational view of the present invention.

FIG. 4 is a left-side elevational view of the present invention.

FIG. 5 is a top plan view of the present invention.

FIG. 6 is a cross-sectional view taken along line 6-6 in FIG. 3, showing the internal structure of the layered bag.

FIG. 7 is a top-front perspective view of the present invention, shown in use as a first aid kit.

FIG. 8 is a flowchart of a method of using the present invention.

### DETAIL DESCRIPTIONS OF THE INVENTION

All illustrations of the drawings are for the purpose of describing selected versions of the present invention and are not intended to limit the scope of the present invention.

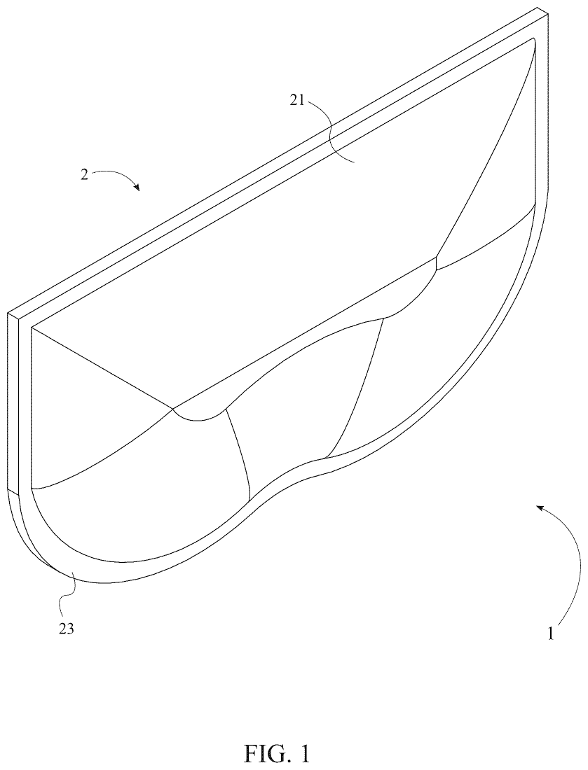

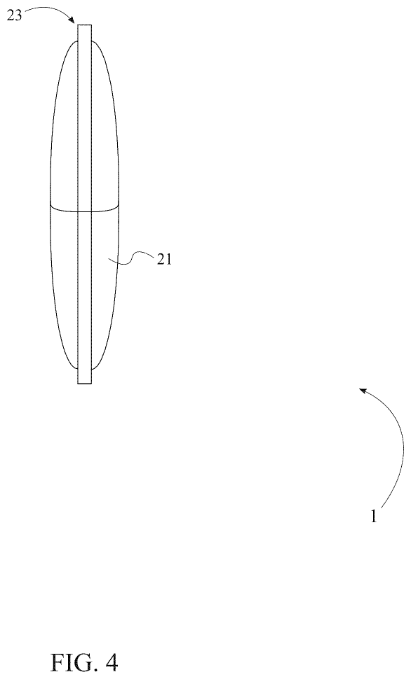

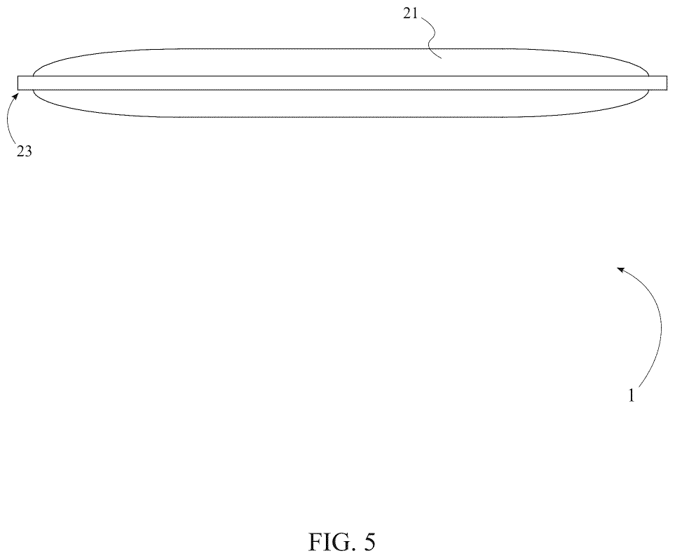

In reference to FIGS. 1-8, the present invention is a system and method for stopping nosebleeds quickly in an enhanced sanitary and hygienic fashion. An objective of the present invention is to provide users with a layered bag that is shaped to fit around the nasal bone of the user. The layered bag provides a means for inducing vasoconstriction of the nasal cavity while protecting the skin tissue from rapid cooling. The device may be stored anywhere for up to two years without the need for a refrigerator. To accomplish these objectives, the system 1 of the present invention comprises a layered bag 2 and a cushion 3. The cushion 3 is sealed within the layered bag 2 and submersed in water. Once the cushion 3 is punctured, an endothermic reaction occurs, cooling the water within the layered bag 2. Thus, the present invention is a system and method for stopping nosebleeds without damaging the epidermis and facilitating proper disposal of contaminated cleaning agents.

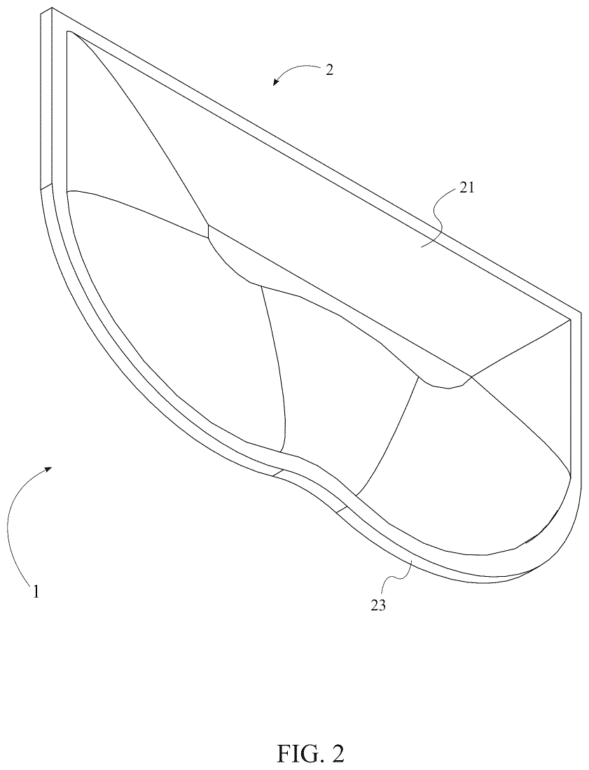

The layered bag 2 is a type of face covering that is shaped to fit the nasal bone. The layered bag 2 comprises an outer covering 21, water 22, and a seal 23. The outer covering 21 is preferably a double-layer fabric material comprising PET (polyethylene terephthalate) and aluminum. This arrangement allows the layered bag 2 to easily stretch and conform to the shape of the nasal bone. As can be seen in FIG. 1 and FIG. 2, the seal 23 is integrated along the perimeter of the outer covering 21. The seal 23 forms a cavity 24 within the outer covering 21 where the water 22 is contained. In a preferred embodiment, the water 22 is a chemically pure water to prevent contamination of the eyes and face in case of potential damage. It is understood that the outer covering 21 can be created in various shapes and sizes based on design, user, and or manufacturing requirements. Moreover, the seal 23 can be formed and perimetrically integrated along the outer covering 21 by way of heat sealing or by any other suitable means based on design, user, and or manufacturing requirements.

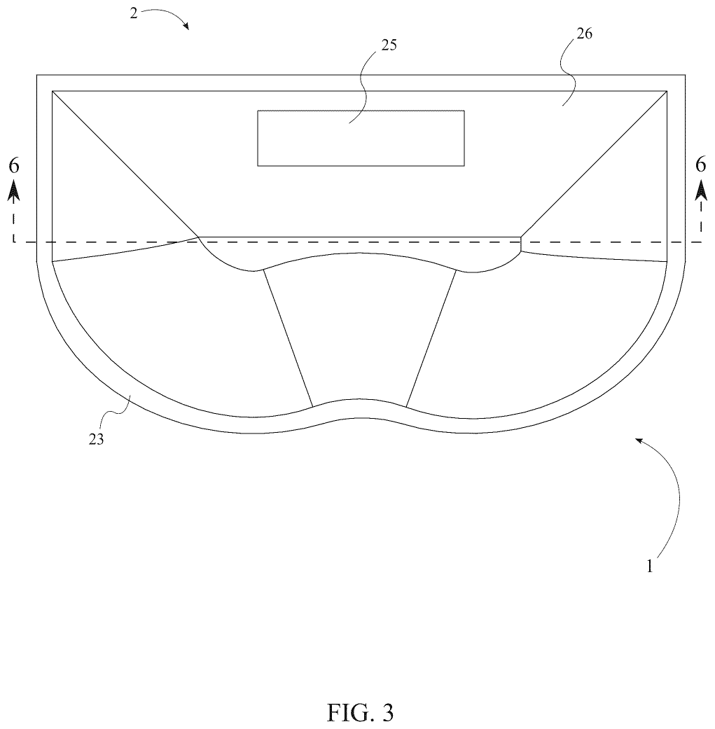

As can be seen in FIG. 3, in a preferred embodiment, the layered bag 2 further comprises an adhesive tape 25. In this embodiment, the adhesive tape 25 is preferably double-sided and made of low heat-conductive material. The adhesive tape 25 serves two functions, namely, (a) for attaching the layered bag 2 to the face and (b) for preventing skin tissue from rapidly cooling and potentially causing frostbite after activation. The adhesive tape 25 is positioned along the outer surface of the layered bag 2. More specifically, the adhesive tape 25 is secured to an exterior surface 26 of the outer covering 21, facing the user.

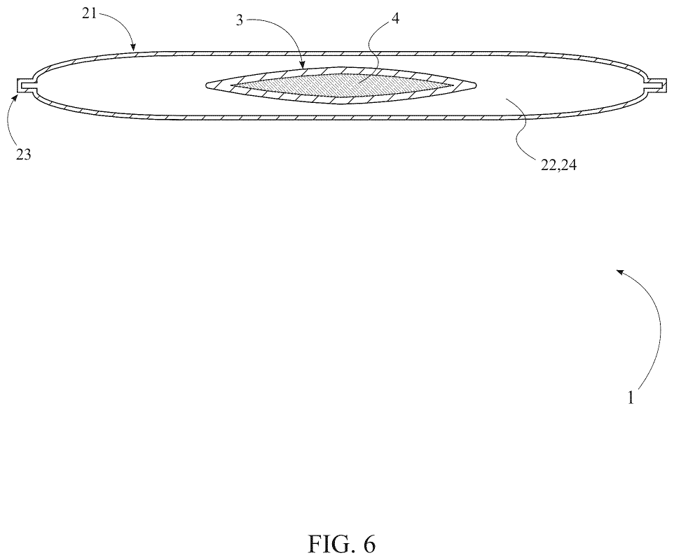

As can be seen in FIG. 6, the cushion 3 and the water 22 are both sealed within the cavity 24, wherein the cushion 3 is submerged in water 22. The cushion 3 is a flexible material, designed to contain and seal a water-reactive substance 4, namely urea, from the water 22. When squeezed, the cushion 3 is designed to rupture and disperse the urea into the surrounding water 22 within the cavity 24. Once the urea 4 is released and mixed with the water 22, an endothermic reaction initiates, thereby cooling the layered bag to −0.5 to −0.6 degrees Celsius. This arrangement provides the optimal temperature not to damage the epidermis and irritate the nasal cavity, especially for children. After activation, the outer covering 21 covers the nasal bone as much as possible, at which point the layered bag 2 conforms to the shape of the nasal bone and is applied to the nasal bone to induce vasoconstriction of the nasal cavity. In the preferred embodiment, the chemical composition of the two substances within the cavity 24 is 45% urea and 55% water. This arrangement ensures the proper temperature drop within the desired amount of time. In other embodiments, the chemical composition of urea and water can vary depending on design, user, and or manufacturing requirements.

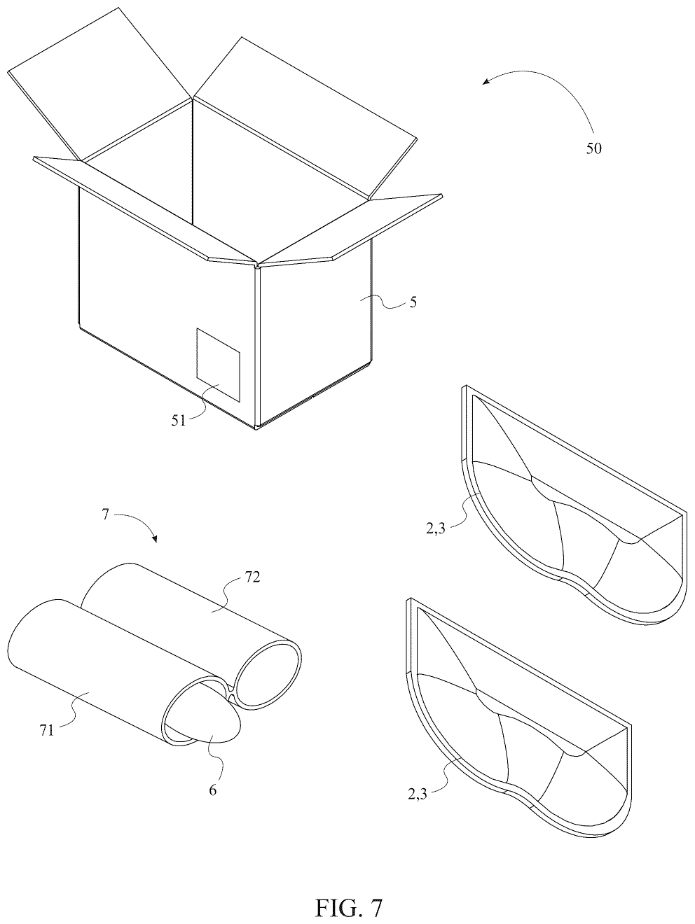

As can be seen in FIG. 7, in the preferred embodiment, the present invention is provided to the user in the form of a first aid kit 50. The first aid kit 50 comprises an information box 5, at least one layered bag 2 with a pre-installed cushion 3, at least one cotton swab 6, and at least one disposable container 7. The contents within the layered bag are non-toxic and non-allergic. Once the layered bag 2 is activated and cooling begins, it is virtually impossible to damage the layered bag and extract the contents. Nevertheless, first aid rules are disclosed along the information box 5 in case of an accident. The cotton swab 6 is used to absorb the blood draining from the nasal cavity. After use, the contaminated cotton swab 6 can be stored and secured in the disposable container 7. Preferably, the disposable container 7 is a bio-safe, two-part paper container comprising a first compartment 71 and a second compartment 72. The first compartment 71 is designed to store a clean cotton swab 6 before use, while the second compartment 72 is designed to store and secure a contaminated cotton swab 6 after use. In addition, a QR code 51 is preferably printed along the information box 5. After scanning the QR code 51 with a smartphone or other compatible device, the user is directed to a URL website with instructions on how to use the first aid kit 50.

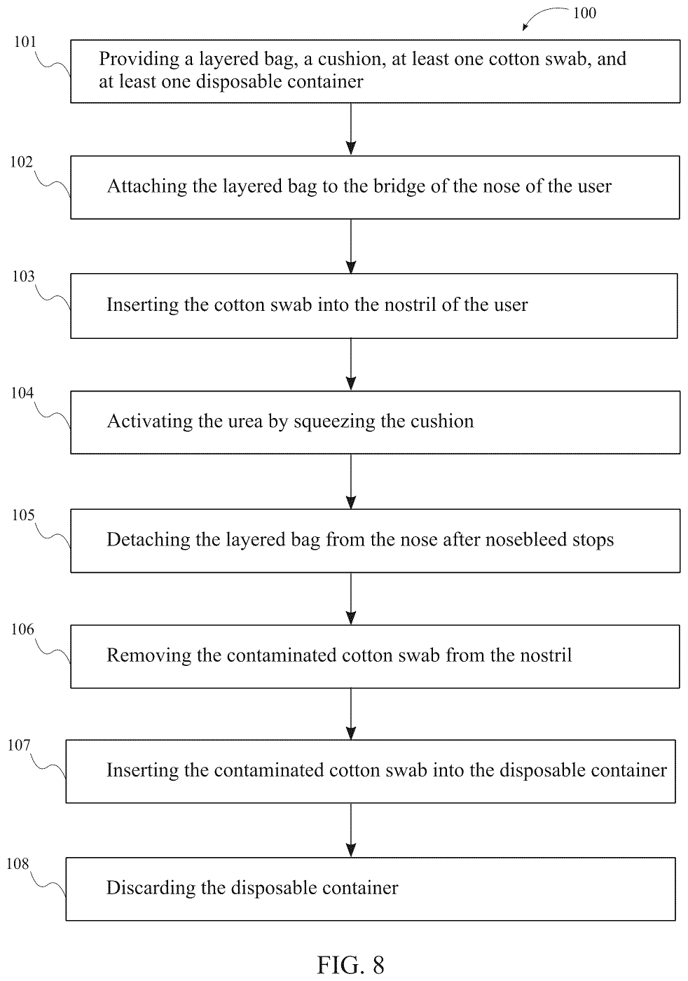

In reference to FIG. 8, a method 100 for using the present invention comprises the following steps. First, at step 101, the method may include providing a layered bag, a cushion, at least one cotton swab, and at least one disposable container. The layered bag comprises an outer covering, water, and a seal, wherein the seal is perimetrically integrated along the outer covering and the cushion is submerged in water and sealed within the cavity. Next, at step 102, the method may include attaching the layered bag to the bridge of the nose of the user. Preferably in this step, the layered bag further comprises an adhesive tape, which helps secure the layered bag to the nose. However, in other embodiments where the layered bag is not configured with an adhesive tape, the layered bag can be secured to the user's nose by any suitable means of attachment, including but not limited to elastic straps or press fit.

Continuing with the method at step 103, the method may include inserting the cotton swab into the nostril of the user. The cotton swab helps absorb blood drained from the nasal cavity. Further, at step 104, the method may include activating the water-reactive substance by squeezing the cushion. Preferably in this step, the water-reactive substance is urea, which causes the layered bag to rapidly cool after the cushion is ruptured by squeezing. The cold surface applied to the nose induces vasoconstriction within the nasal cavity, which in turn, reduces the amount of time needed to stop the nosebleed. Once the nosebleed stops, the next step 105 may include detaching the layered bag from the nose and step 106 may include removing the contaminated cotton swab from the nostril.

For proper disposal of biohazardous material, the next step 107 may include inserting the contaminated cotton swab into the disposable container. Preferably in this step, the disposable container is in the form of a bio-safe, two-part paper container comprising a first compartment and a second compartment. Here, the contaminated cotton swab can be inserted into the second compartment. Next, at step 108, the method may include discarding the disposable container.

Although the invention has been explained in relation to its preferred embodiment, it is to be understood that many other possible modifications and variations can be made without departing from the spirit and scope of the invention.

What is claimed is:

1. A system for treating a nosebleed, the system comprising:
a layered bag;
a cushion;
the layered bag comprising an outer covering, water, and a seal;
the outer covering shaped to surround the nasal bone of the user;
the seal being perimetrically integrated along the outer covering;
the seal forming a cavity within the outer covering;
the cushion comprising a water-reactive substance;
the cushion and the water being sealed within the cavity, wherein the cushion is submerged in the water; and
the cushion being configured to rupture and activate the water-reactive substance by squeezing the cushion.

2. The system as claimed in claim 1 comprising:
the outer covering being a double-layer fabric material comprising polyethylene terephthalate and aluminum; and
the layered bag capable of conforming to the shape of the nasal bone after activation.

3. The system as claimed in claim 1 comprising:
the water-reactive substance being urea; and
the layered bag being configured to rapidly cool via endothermic reaction after activation.

4. The system as claimed in claim 3, wherein a chemical composition within the cavity is 45% urea and 55% water.

5. The system as claimed in claim 3 comprising:
the layered bag further comprising an adhesive tape; and
the adhesive tape being attached to an exterior surface of the outer covering, facing the user.

6. The system as claimed in claim 5 comprising:
the adhesive tape being made of low heat-conductive material; and
the adhesive tape being configured to prevent skin tissue from rapidly cooling after activation.

7. The system as claimed in claim 3 comprising:
at least one cotton swab; and
the cotton swab being configured to absorb blood drained from the nasal cavity of the user.

8. The system as claimed in claim 7 comprising:
at least one disposable container; and
the disposable container being configured to store and secure the contaminated cotton swab after use.

9. The system as claimed in claim 8, wherein the disposable container comprises a first compartment and a second compartment.

10. A system for treating a nosebleed, the system comprising:
a layered bag;
a cushion;
at least one cotton swab;
at least one disposable container;
the layered bag comprising an outer covering, water, a seal, and an adhesive tape;
the adhesive tape being attached to an exterior surface of the outer covering, facing the user;
the outer covering shaped to surround the nasal bone of the user;
the seal being perimetrically integrated along the outer covering;
the seal forming a cavity within the outer covering;
the cushion comprising urea;
the cushion and the water being sealed within the cavity, wherein the cushion is submerged in the water;
the cushion being configured to rupture and activate the urea by squeezing the cushion;
the layered bag being configured to rapidly cool via endothermic reaction after activation;
the cotton swab being configured to absorb blood drained from the nasal cavity of the user; and
the disposable container being configured to store and secure the contaminated cotton swab after use.

11. The system as claimed in claim 10 comprising:
the outer covering being a double-layer fabric material comprising polyethylene terephthalate and aluminum; and
the layered bag capable of conforming to the shape of the nasal bone after activation.

12. The system as claimed in claim 10, wherein a chemical composition within the cavity is 45% urea and 55% water.

13. The system as claimed in claim 10 comprising:
the adhesive tape being made of low heat-conductive material; and
the adhesive tape being configured to prevent skin tissue from rapidly cooling after activation.

14. The system as claimed in claim 10, wherein the at least one disposable container comprises a first compartment and a second compartment.

15. A method for treating a nosebleed, the method comprising:
providing a layered bag, a cushion, at least one cotton swab, and at least one disposable container;
the layered bag comprising an outer covering, water, and a seal;
the seal being perimetrically integrated along the outer covering;
the seal forming a cavity within the outer covering;
the cushion comprising urea; and
the cushion and the water being sealed within the cavity, wherein the cushion is submerged in the water;
attaching the layered bag to the bridge of the nose of the user;
inserting the cotton swab into the nostril of the user;
activating the urea by squeezing the cushion;
detaching the layered bag from the nose after the nosebleed stops;
removing the contaminated cotton swab from the nostril;
inserting the contaminated cotton swab into the disposable container; and
discarding the disposable container.

16. The method as claimed in claim 15 comprising:
the outer covering being a double-layer fabric material comprising polyethylene terephthalate and aluminum; and
the layered bag capable of conforming to the shape of the nasal bone after activation.

17. The method as claimed in claim 15, wherein a chemical composition within the cavity is 45% urea and 55% water.

18. The method as claimed in claim 15 comprising:
the layered bag further comprising an adhesive tape; and
the adhesive tape being attached to an exterior surface of the outer covering, facing the user.

19. The method as claimed in claim 18 comprising:
the adhesive tape being made of low heat-conductive material; and
the adhesive tape being configured to prevent skin tissue from rapidly cooling after activation.

20. The method as claimed in claim 15, wherein the disposable container comprises a first compartment and a second compartment.
